(19) United States
(12) Patent Application Publication
(10) Pub. No.: US 2011/0008447 A1
(43) Pub. Date: Jan. 13, 2011

(54) CARRIER COMPRISING NANODIAMOND

(75) Inventors: Jui-I Chao, Hsinchu (TW); Chin-Piao Chen, Hsinchu (TW); Chia-Liang Cheng, Hsinchu (TW)

Correspondence Address: EDWARDS ANGELL PALMER & DODGE LLP, P.O. BOX 55874, BOSTON, MA, 02205, US

(73) Assignee: NATIONAL CHIAO TUNG UNIVERSITY, Hsinchu (TW)

(21) Appl. No.: 12/574,958

(22) Filed: Oct. 7, 2009

(30) Foreign Application Priority Data
Jul. 13, 2009 (TW) 98123574

(51) Int. Cl. A61K 9/14; A61K 31/5377; A61K 31/4045; A61K 38/02; A61K 31/7088; A61P 35/00

(52) U.S. Cl. 424/489; 514/234.5; 514/414; 514/44 R; 514/19.3; 514/19.4

(57) ABSTRACT

The present invention provides a carrier including a nanodiamond (ND) particle and a linker covalently bound to the ND particle, in which the linker is presented by the formula: —R¹—O(R²)ₘ-Q-. In addition, the present invention further provides a carrier having an active unit covalently bound to the linker, in which the active unit is a drug, a vitamin or a biological molecule.

### FIELD OF INVENTION

The present invention relates to a carrier including a nanodiamond particle, and more particularly, to a carrier including a nanodiamond particle and a linker. Further, the present invention relates to a carrier including a nanodiamond particle, a linker and an active unit.

### BACKGROUND OF THE INVENTION

Cancer is current main cause of human death. The current cancer therapies include surgical operations, radiation therapies, chemotherapies, etc. For late-stage cancer patients, chemotherapies should be taken and followed by further therapies. However, the medication circulated in body usually has poor stability and is not easy to be taken into cells. Further, anti-cancer drugs have non-specific toxicity to normal cells and healthy tissues, thereby reducing therapy effects thereof. Hence, it is an urgent issue to develop novel therapy for cancers.

It has been reported that the anti-cancer drugs or biological molecules can be bounded with nanoparticles for enhancing stability thereof and further providing better cancer therapies. In the research of nanoparticles, it has been reported that carbon nanotubes and carbon-60 have more bio-toxicity; however, it has been proven that nanodiamond (ND) induces no significant cellular toxicity in human lung cells (K. K. Liu, C. L. Cheng, C. C. Chang, and J. I. Chao, Nanotechnology, 2007, 18, 325102), neuron cells (A. M. Schrand, H. Huang, C. Carlson, J. J. Schlager, E. Omacr Sawa, S. M. Hussain, L. Dai, J. Phys. Chem. B 2007, 111, 2-7), kidney cells (S. J. Yu, M. W. Kang, H. C. Chang, K. M. Chen, Y. C. Yu, J. Am. Chem. Soc. 2005, 127, 17604-17605; T. Lechleitner, F. Klauser, T. Seppi, J. Lechner, P. Jennings, P. Perco, B. Mayer, D. Steinmuller-Nethl, J. Preiner, P. Hinterdorfer, M. Hermann, E. Bertel, K. Pfaller, W. Pfaller, Biomaterials 2008, 29, 4275-4284) and cervical cells (I. P. Chang, K. C. Hwang, C. S. Chiang, J. Am. Chem. Soc. 2008, 130, 15476-15481). Nanodiamond has no toxicity and better biocompatibility than other nanoparticles, and is thus more suitable to be used in biomedical field. Further, nanodiamond has fluorescent property, so that nanodiamond can be used in biolabeling, detection and trace while being connected to drugs or biomolecules. Therefore, nanodiamond has great potential in biomedical field.

It is one application of nanodiamond in biomedical field that drugs are bound to the surface of nanodiamond. Nanodiamond powder is formed by detonation synthesis, and has nanodiamond core covered by one or more carbon and amorphous carbon. Also, the surface of nanodiamond is coated by various functional groups including carboxyl, lactone, ketone, hydroxyl, alkyl, etc. Since the surface of nanodiamond has carboxyl and/or carboxylic acid, nanodiamond particles are suitable to be a substrate of functionalized nanomaterials.

The surface of nanodiamond is easy to be modified, such that nanodiamond particles are valuable nanomaterials in recent years. There are two modifications on the surface of nanodiamond particles including covalent bonding and non-covalent bonding. Chao et al. (J. I. Chao, E. Perevedentseva, P. H. Chung, K. K. Liu, C. Y. Cheng, C. C. Chang, C. L. Cheng, Biophys. J. 2007, 93, 2199-2208) and Liu et al. (K. K. Liu, M. F. Chen, P. Y. Chen, T. J. F. Lee, C. L. Cheng, C. C. Chang, Y. P. Ho, J. I. Chao, Nanotechnology 2008, 19, 205102) have disclosed lysozyme and alpha-bungarotoxin bound to carboxylated surface of nanodiamond via adsorption and maintaining protein activity thereof, respectively, for performing biological reactions. Huang et al. (H. Huang, E. Pierstorff, E. Osawa, D. Ho, Nano Lett. 2007, 7, 3305-3314) have disclosed nanodiamond hydrogels absorbing doxorubicin via non-covalent bonding for delivering anti-cancer drugs. Huang et al. (L. C. Huang, H. C. Chang, Langmuir 2004, 20, 5879-5884) have also disclosed cytochrome c absorbed on the surface of nanodiamond via non-covalent bonding. However, the above drug adsorption via non-covalent bonding results in that when the drug is delivered in body, the drug may be dissociated, so as to cause instability of the drug circulated in body.

Further, U.S. Patent Application Publication Nos. 2006/0269467 and 2005/0158549 have disclosed methods for preparing nanodiamond with halogen gas or halogen acid. However, there are safety concerns about handling halogen materials. For example, when forming functional groups on nanodiamond, toxic gas including halogens may be produced. In addition, Ushizawa et al. (Koichi Ushizawa et al., Chem. Phys. Lett. 2002, 351, 105-108) have disclosed DNA reacting with COCl on the surface of nanodiamond via covalent bonding. However, nanodiamond is a nanoparticle much larger than biological molecules, and thus has steric hinderance disfavoring chemical reactions, so that it is hard to increase derivatives of the biological molecules. Regarding the functionalized nanodiamond made by Ushizawa et al., —COCl group is in contact with the nanodiamond and fails to have effective reaction with DNA, such that DNA cannot be effectively formed on the surface of nanodiamond.

Accordingly, it is an urgent issue to provide drugs or biological molecules formed with nanodiamond via covalent bonding applicable in the biomedical field, wherein the stability and activity of the drugs or biological molecules circulated in body are maintained.

### SUMMARY OF THE INVENTION

The present invention provides a carrier having nanodiamond covalently bound with an active unit, thereby eliminating dissociation of the active unit due to a physical treatment (such as washing). Further, the present invention provides a carrier having a linker for avoiding low derivatives resulting from stereo block and facilitating applications in the biomedical field.

It is an aspect of the present invention to provide a carrier including a nanodiamond (ND) particle and a linker covalently bound to the nanodiamond particle.

In accordance with the present invention, the linker is covalently bound to a surface of the nanodiamond particle, and the linker is presented as —R¹—O(R²)ₘ-Q-, wherein R¹ and R² are independently selected from the group consisting of a covalent bond, C₁₋₂₀alkyl, C₂₋₂₀alkenyl, C₂₋₂₀alkynyl, C₁₋₂₀alkoxy, C₁₋₂₀alkylthio and C₁₋₂₀alkylamino, in which the C₁₋₂₀alkyl, C₂₋₂₀alkenyl, C₂₋₂₀alkynyl, C₁₋₂₀alkoxy, C₁₋₂₀alkylthio and C₁₋₂₀alkylamino are optionally substituted by at least one selected from the group consisting of hydroxyl, halogen, cyano, nitro, carboxyl, C₁₋₂₀alkyl, C₂₋₂₀alkenyl, C₂₋₂₀alkynyl, C₁₋₂₀alkoxy, C₂₋₂₀alkyl ether, C₃₋₂₀alkyl ketone, C₁₋₂₀alkylthio, amino, mono-(C₁-C₂₀alkyl)amino, di-(C₁-C₂ₒalkyl)amino, haloC₁₋₂₀alkyl, haloC₁₋₂₀alkoxy, C₁₋₂₀alkanoyl, C₂₋₂₀alkanoyloxy, C₁₋₂₀alkoxycarbonyl, amido (—CONH₂), mono-(C₁₋₂₀alkyl)aminocarbony, di-(C₁₋₂₀alkyl)aminocarbonyl, sulfonylamino (—SO₂NH₂), mono-(C₁₋₂₀alkyl)sulfonylamino and di-(C₁₋₂₀alkyl)sulfonylamino; Q is hydroxyl, amino, carbonyl, acyl, keto, carboxyl, halogen, cayno, thio, C₁₋₂₀alkyl, C₁₋₂₀alkoxy, C₂₋₂₀alkenyl, C₂₋₂₀alkynyl, C₆₋₁₆aryl, azide, aldehyde, thiocyano, CO₂(R³)ₙ, CO(R⁴)ₙ, NHR⁵, N(R⁶)ₙ, SR⁷ or O(R⁸)ₙ, in which R³, R⁴, R⁵, R⁶, R⁷ and R⁸ are independently selected at each occurrence from the group consisting of halogen, amino, carbonyl, acyl, keto, carboxyl, phenylsulfonyl, sulfonyl, C₁₋₂₀alkyl, C₂₋₂₀alkenyl, C₂₋₂₀alkynyl, C₁₋₂₀alkoxy and C₆₋₁₆aryl, and n at each occurrence is an integer from 1 to 20, preferably an integer from 1 to 16, and more preferably an integer from 1 to 12; and m at each occurrence is an integer from 1 to 20, preferably an integer from 1 to 16, and more preferably an integer from 1 to 12.

In accordance with the present invention, R¹ and R² of the linker are optionally substituted methyl, and the carrier includes one or more linkers bound to the nanodiamond particle.

In accordance with the present invention, the active unit can be a drug, vitamin or biological molecule. The drug can be an anti-cancer drug, and preferably an anti-microtubule agent, and more preferably taxol, Iressa or Sutent. The vitamin can be vitamin K3, vitamin C, vitamin D, vitamin E, vitamin H or vitamin B7. The biological molecule can be nucleic acid, peptide, protein or derivatives thereof, wherein the nucleic acid is DNA or RNA. According to an embodiment of the present invention, the active unit is an optical isomer.

It is an another aspect of the present invention to provide a method for preparing the above carrier, including at least the steps of: providing a nanodiamond particle; performing acidification and oxidation on the nanodiamond particle to form a first intermediate; performing reduction on the first intermediate to form a second intermediate; performing alkylation on the second intermediate to form a third intermediate; and performing an reaction of the third intermediate and a substituent. The acidification comprises a treatment with an inorganic acid, such as hydrochloric acid, nitric acid, sulfuric acid or a mixed solution thereof, more preferably a mixed solution of hydrochloric acid and nitric acid or a mixed solution of nitric acid and sulfuric acid. The acidification comprises a treatment with aluminum hydride agent or boron hydride agent, preferably lithium aluminum hydride (LAH) or sodium borohydride (NaBH₄). The alkylation comprises substituting any functional group of the second intermediate with C₁₋₂₀alkyl.

In the method of the present invention, the substituent comprises hydroxyl, amino, carbonyl, acyl, keto, carboxyl, halogen, cayno, thio, C₁₋₂₀alkyl, C₁₋₂₀alkoxy, C₂₋₂₀alkenyl, C₂₋₂₀alkynyl, C₆₋₁₆aryl, azide, aldehyde, thiocyano, CO₂(R³)ₙ, CO(R⁴)ₙ, NHR⁵, N(R⁶)ₙ, SR⁷ or O(R⁸)ₙ, in which R³, R⁴, R⁵, R⁶, R⁷ and R⁸ are independently selected at each occurrence from the group consisting of halogen, amino, carbonyl, acyl, keto, carboxyl, phenylsulfonyl, sulfonyl, C₁₋₂₀alkyl, C₂₋₂₀alkenyl, C₂₋₂₀ alkynyl, C₁₋₂₀alkoxy and C₆₋₁₆aryl, and n at each occurrence is an integer from 1 to 20, preferably an integer from 1 to 16, and more preferably an integer from 1 to 12.

In accordance with the present invention, the substituent is covalently bound to the active unit. In accordance with the present invention, the active unit can be a drug, vitamin or biological molecule. The drug can be an anti-cancer drug, and preferably an anti-microtubule agent, and more preferably taxol, Iressa or Sutent. The vitamin can be vitamin K3, vitamin C, vitamin D, vitamin E, vitamin H or vitamin B7. The biological molecule can be nucleic acid, peptide, protein or derivatives thereof, wherein the nucleic acid is DNA or RNA. According to an embodiment of the present invention, the active unit is an optical isomer.

It is another aspect of the present invention to provide a kit, including above carrier and a reagent, wherein the reagent includes a reagent needed in PCR reaction, a reagent needed in agarose gel electrophoresis, and a reagent needed in immunoactivity reaction.

It is another aspect of the present invention to provide a method for in vitro detecting a biological molecule by using the above carrier, wherein the biological molecule includes a nucleic acid, peptide, protein and derivatives thereof.

It is another aspect of the present invention to provide a use of the above carrier for preparing a drug for treating a cancer, wherein the cancer is lung cancer, breast cancer, colorectal cancer, cervical cancer, bladder cancer or other cancers.

### BRIEF DESCRIPTION OF THE DRAWINGS

FIG. 1 shows FTIR spectrum of ND-linker (5) in scheme 1 according to the present invention;

FIG. 2 shows FTIR spectrum of ND-linker (7) in scheme 1 according to the present invention;

FIG. 3 shows FTIR spectrum of ND-linker (21) in scheme 4 according to the present invention;

FIG. 4 shows FTIR spectrum of ND-linker-vitamin K3 (23) in scheme 4 according to the present invention;

FIG. 5 shows FTIR spectrum of ND-linker-peptide (27) in scheme 5 according to the present invention;

FIG. 6 shows FTIR spectrum of (31) in scheme 6 according to the present invention;

FIGS. 7A-7H show FTIR spectra of ND-linkers (41) and (42) in scheme 9 according to the present invention;

FIG. 8A and FIG. 8B show the schematic view of the carrier having taxol and spectra thereof, respectively, wherein the spectra are deuterated CD unique IR spectra to confirm the derivates in all steps bound to the nanodiamond according to the present invention;

FIG. 9A and FIG. 9B show the morphology and size of nanodiamond and the carrier having taxol by AFM and SEM, respectively, according to the present invention;

FIG. 10A and FIG. 10B show fluorescence indicating mitosis of human A549 lung cancer cells inhibited by the carrier having taxol by confocal microscope according to the present invention;

FIG. 11 shows ND-linker-taxol stabilizing polymerization of microtubles in human A549 lung caner cells so as to inhibit separation of chromosomes by using confocal microscope according to the present invention;

FIG. 12A and FIG. 12B show the carrier having taxol inducing apoptosis of human A549 lung cancer cells and inhibiting mitosis of cancer cells according to the present invention;

FIG. 13 shows taxol losing activity after being treated with 1M NaOH and failing to inhibiting cancer cells, proving that taxol bound on nanodiamond of the present invention indeed has anti-cancer activity;

FIG. 14 shows that the nanodiamond particle has no cytotoxicity;

FIGS. 15A-E show the carrier having taxol inducing cell death of human A549 lung cancer cells, RKO cells (colorectal cancer cells), HCT116 cells (colon cancer cells), BFTC905 (bladder cancer cells) and HeLa cells (cervical cancer cells) in a concentration-dependent manner; and

FIGS. 16A and 16B respectively show viability and the Z-axial scanning image of lung cancer cells treated with ND-taxol, which was treated with NaOH.

### DETAILED DESCRIPTION OF THE PREFERRED EMBODIMENTS

Functional groups of the present invention are generally described using standard nomenclature. It is to be understood that the linker is described herein using a general formula, such as R¹ to R⁸ and Q. Unless otherwise specified, each variable within such a formula is defined at each occurrence independently of other variables.

The term “alkyl” of the present invention is intended to include branched, straight-chain and circular saturated aliphatic hydrocarbon groups. Alkyl groups can be bound to an atom of a molecule via suitable moiety. The term “alkyl” indicates a group having from 1 to 20 carbon atoms (C₁₋₂₀alkyl), from 1 to 16 carbon atoms (C₁₋₁₆alkyl), or from 1 to 12 carbon atoms (C₁₋₁₂alkyl), such as methyl, ethyl, n-propyl, isopropyl, n-butyl, sec-butyl, tert-butyl, pentyl, 2-pentyl, isopentyl, neopentyl, hexyl, 2-hexyl, 3-hexyl, 3-methylpentyl, cyclopropyl, cyclopropylmethyl, cyclopentyl, cyclopentylmethyl, cyclohexyl and cycloheptyl. In certain embodiments, alkyl group is preferably branched or straight-chain. In some embodiments of the present invention, alkyl group specifically indicates, for example, C₁₋₂₀alkylthio, referring to C₁₋₂₀alkyl group substituted by thio group.

“Alkenyl” refers to a straight or branched hydrocarbon chain comprising one or more unsaturated carbon-carbon bonds. Alkenyl groups include C₂₋₂₀alkenyl groups which have from 2 to 20 carbon atoms (C₂₋₂₀alkenyl), from 2 to 16 carbon atoms (C₂₋₁₆alkenyl) and from 2 to 12 carbon atoms (C₂₋₁₂alkenyl), respectively, such as ethenyl, propenyl or isopropenyl. “Alkynyl” refers to straight or branched hydrocarbon chains comprising one or more triple carbon-carbon bonds. Alkynyl groups include C₂₋₂₀alkynyl, C₂₋₁₆alkynyl and C₂₋₁₂alkynyl groups, which have from 2 to 20, from 2 to 16 and from 2 to 12 carbon atoms, respectively. In certain embodiments, alkenyl groups and alkynyl groups are preferably straight or branched chains.

“Alkoxy” represents an alkyl group as defined above with the indicated number of carbon atoms attached through an oxygen bridge. Alkoxy groups include C₁₋₂₀alkoxy, C₁₋₁₆alkoxy and C₁₋₁₂alkoxy, which have from 1 to 20, from 1 to 16 and from 1 to 12 carbon atoms, respectively. Examples of alkoxy groups include methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Similarly, the term “alkylthio” refers to an alkyl group attached via a thioether linkage. Preferably, alkoxy groups and alkylthio groups are alkyl groups attached via a heteroatom bridge.

The term “alkanoyl” refers to an acyl group in a linear or branched arrangement (such as —(C═O)-alkyl). Alkanoyl groups include C₂₋₂₀alkanoyl, C₂₋₁₆alkanoyl and C₂₋₁₂alkanoyl groups, which have from 2 to 20, from 2 to 16, and from 2 to 12 carbon atoms, respectively. “C₁alkanoyl” refers to —(C═O)—H, which is encompassed by the term “C₁₋₂₀alkanoyl”.

“Alkyl ketone” has a ketone group having carbon atoms in a linear, branched or circular arrangement. C₃₋₂₀alkyl ketone, C₃₋₁₆alkyl ketone and C₃₋₁₂alkyl ketone have from 3 to 20, from 3 to 16, and from 3 to 12 carbon atoms, respectively. For example, C₃alkyl ketone has the structure —CH₂—(C═O)—CH₂—.

The term “alkyl ether” refers to a linear or branched ether substituent linked via a carbon-oxygen-carbon bond. Alkyl ether groups include C₂₋₂₀alkyl ether, C₂₋₁₆alkyl ether and C₂₋₁₂alkyl ether groups, which have 2 to 20, from 2 to 16, and from 2 to 12 carbon atoms, respectively. For example, C₂alkyl ether has the structure —CH₂—O—CH₂—. For example, the branched alkyl ether has the structure —C(CH₃)₂CH₂—O—CH₃.

The term “alkoxycarbonyl” refers to an alkoxy group linked via a carbonyl (i.e., a group having the general structure-C(═O)—O-alkyl). Alkoxycarbonyl groups include C₂₋₂₀alkoxycarbony, C₂₋₁₆alkoxycarbony, and C₂₋₁₂alkoxycarbonyl groups, which have from 2 to 20, from 2 to 16, and 2 to 16 carbon atoms, respectively. “C₁alkoxycarbonyl” refers to —C(═O)—OH, and is encompassed by “C₁₋₂₀alkoxycarbonyl”.

“Alkanoyloxy” as used herein refers to an alkanoyl group linked via an oxygen bridge (i.e., a group having the general structure —OC(═O)-alkyl). Alkanoyloxy groups include C₂₋₂₀alkanoyloxy, C₂₋₁₆alkanoyloxy, and C₂₋₁₂alkanoyloxy groups, which have from 2 to 20, from 2 to 16, and 2 to 12 carbon atoms, respectively.

“Alkylamino” refers to a secondary or tertiary amine having the general structure —NH-alkyl or —N(alkyl)(alkyl), wherein each alkyl may be the same or different. Such groups include, for example, mono- and di-(C₁₋₂₀alkyl) amino groups, in which each alkyl may be the same or different and may contain from 1 to 20 carbon atoms.

The term “amido” refers to an amide group (i.e., —(C═O)NH₂). Mono- or di-(C₁₋₂₀alkyl)amido means one or two hydrogen atoms of amido group is substituted by C₁₋₂₀alkyl, wherein if both hydrogen atoms are substituted, the C₁₋₂₀alkyl groups can be the same or different.

The term “halogen” indicates fluorine, chlorine, bromine, or iodine. “Haloalkyl” refers to both branched and straight-chain saturated aliphatic hydrocarbon groups having the specified number of carbon atoms, substituted with 1 or more halogen atoms (i.e., haloC₁₋₈alkyl group has from 1 to 8 carbon atoms; haloC₁₋₆alkyl group has from 1 to 6 carbon atoms). Examples of haloalkyl include, but are not limited to, mono-, di- or trifluoromethyl; mono-, di- or trichloromethyl; mono-, di-tri-, tetra- or penta-fluoroethyl; mono-, di-, tri-, tetra- or penta-chloroethyl; and 1,2,2,2-tetrafluoro-1-trifluoromethyl-ethyl. “Haloalkoxy” indicates a haloalkyl group as defined above attached through an oxygen bridge. HaloC1-8alkoxy group has from 1 to 8 carbon atoms.

As used herein, the term “aryl” indicates aromatic groups containing only carbon in the aromatic ring(s). In addition to aromatic rings, aryl groups can include non-aromatic rings. C₆₋₁₆aryl groups have from 6 to 16 carbon atoms. Specifically preferred aryl groups include phenyl, napthyl (such as 1-naphthyl and 2-naphthyl), biphenyl, tetralyl and indenyl.

A dash (“-”) that is between two letters or symbols is used to indicate a point of attachment for a substituent. For example, —CONH₂ is attached through the carbon atom.

A “substituent” as used herein, refers to a molecular moiety that is covalently bonded to an atom within a molecule of interest. For example, a “ring substituent” may be a moiety such as a halogen, alkyl group, haloalkyl group or other substituent discussed herein that is covalently bonded to an atom (preferably a carbon or nitrogen atom) that is a ring member. The term “substituted” as used herein means that any one or more hydrogens on the designated atom is replaced with a selection from the indicated substituents, provided that the designated atom's normal valence is not exceeded, and that the substitution results in a stable compound (i.e., a compound that can be isolated, characterized and tested for biological activity).

The phrase “optionally substituted” indicates that a group may either be unsubstituted or substituted at one or more of any of the available positions, typically 1, 2, 3, 4, or 5 positions, by one or more suitable substituents such as those (which can be the same or different) disclosed herein. Suitable substituents include, for example, hydroxy, halogen, cyano, nitro, carboxy, C₁₋₂₀alkyl, C₂₋₂₀alkenyl, C₂₋₂₀alkyyl, C₁₋₂₀alkoxy, C₂₋₂₀alkyl ether, C₃₋₂₀alkyl ketone, C₁₋₂₀alkylthio, amino, mono-(C₁-C₂₀alkyl)amino, di-(C₁-C₂₀alkyl)amino, haloC₁₋₂₀alkyl, haloC₁₋₂₀alkoxy, C₁₋₂₀alkanoyl, C₂₋₂₀ alkanoyloxy, C₁₋₂₀ alkoxycarbonyl, amido (—CONH₂), mono-(C₁₋₂₀alkyl)aminocarbonyl, di-(C₁₋₂₀alkyl)aminocarbonyl, sulfonylamino (—SO₂NH₂), mono-(C₁₋₂₀alkyl)sulfonylamino and di-(C₁₋₂₀alkyl)sulfonylamino. Optional substitution may also be indicated by the phrase “substituted with from 0 to Z substituents”, in which Z is the maximum number of substituents. Certain groups provided herein are optionally substituted with from 0 to 2, 0 to 3 or 0 to 4 independently selected substituents (i.e, unsubstituted or substituted by up to the maximum number of substituents).

The term “isomer” refers to compounds with the same molecular formula, but different structure. According to the arrangement or three dimensional positions of atoms, isomers can be divided into structural isomers and stereoisomers. Stereoisomers include geometric isomers, enantiomers and diastereomers. Enantiomers have optical activity as being able to rotate plane-polarized light, and are thus also called optical isomers. For stereoisomers, the specific arrangement of atoms is named as configuration, and enantiomers are two compounds with opposite configurations. The R/S system is a nomenclature system for denoting enantiomers with different configurations.

The term “acidification” refers to performing a treatment with inorganic acid on nanodiamond, wherein the treatment with inorganic acid includes a treatment with hydrochloric acid, nitric acid, sulfuric acid or a mixed solution thereof, or a treatment with alkaline reagent and then while performing acidification, washing the solution into weak acidic solution by using water.

The term “reduction” refers to a treatment with a reducing agent such as aluminum hydride agent or boron hydride agent for reducing carboxyl group, lactone or keto group on nanodiamond to hydroxyl group, wherein the aluminum hydride agent and boron hydride agent include lithium aluminum hydride (LAH) or sodium borohydride (NaBH₄), respectively.

The term “alkylation” refers to substituting any groups of the above identified groups with the above identified alkyl groups.

Microtubules are important targets to be observed in cancer therapy. Paclitaxel or taxol is the most common clinical anti-cancer drug, especially for lung cancer, breast cancer, colon cancer, cervical cancer, bladder cancer, etc. The molecular mechanism of taxol is to stabilize microtubules so as to inhibit mitosis of cancer cells and further to induce apoptosis of cancer cells.

Iressa is a target therapy drug for non-small cell lung cancer. Lung cancer cells produce excess epidermal growth factor receptors, resulting in rapid growth, transformation and drug-resistance of cancer cells. Iressa is an inhibitor of epidermal growth factors, and capable of treating cancers by inhibiting growth and transformation of cancer cells.

Sutent is capable of inhibiting PDGF-R and VEGG-R, and inhibiting proliferation of cancer cells and angiogenesis, so as to inhibit growth and transformation of cancer cells.

The invention has been described using following embodiments. However, it is to be understood that the scope of the invention is not limited to the disclosed embodiments.

### Embodiments

In the following embodiments, taxol was purchased from Tokyo Chemical Industry Co. Ltd., Japan, and succinic anhydride was purchased from Acros Organics (Geel, Belgium). Nanodiamond with diameter of from 3 to 5 nm was purchased from Nanostructured and Amorphous Materials Inc. (Houston, Tex.). 3-(4, 5-Dimethyl-2-thiazolyl)-2,5-diphenyl-2H-tetrazolium bromide (MTT) and Cy3-labeled mouse anti-β-microtubulin (c-4585) were purchased from Sigma Chemical Co. (St. Louis, Mo.). Fourier Transform Infrared Spectroscopy (FTIR) is Perkin Elmer Paragon 1000 FTIR Spectroscopy.

### Embodiment 1: Binding Taxol to Nanodiamond with Linkers

Scheme 1 shows the steps of synthesizing nanodiamond having linkers and binding taxil to nanodiamond. a. Nanodiamond (1) is acidified with HCl/HNO₃ and further oxidized to form (2); b. (2) is reduced by LAH to form (3); c. (3) is modified with a alkyl group to form (4), and the alkyl group is further derivated; d. THP (tetrahydropyranyl) protection group at the end is cleaved to form (5); e. The terminal of (5) is converted to methanesulfonate so as to form (6); and f. The terminal of (6) is converted to —NH₂ group so as to form (7). Then, a reaction of (7) and an active unit is performed, such that the active unit is linked to nanodiamond. Take nanodiamond linked with taxol as an example. g. A reaction of taxol and succinic anhydride is performed to form a product, which then reacts with (7), such that a nanodiamond particle (9) covalently bound with taxol is obtained. The following reaction is further performed on (9). h. (9) is treated with 1M NaOH, so as to remove taxol and obtain (10). In this embodiment, D is used for replacing H (such as d-8, d-9 and d-10), so as to analyze spectra the carrier having taxol.

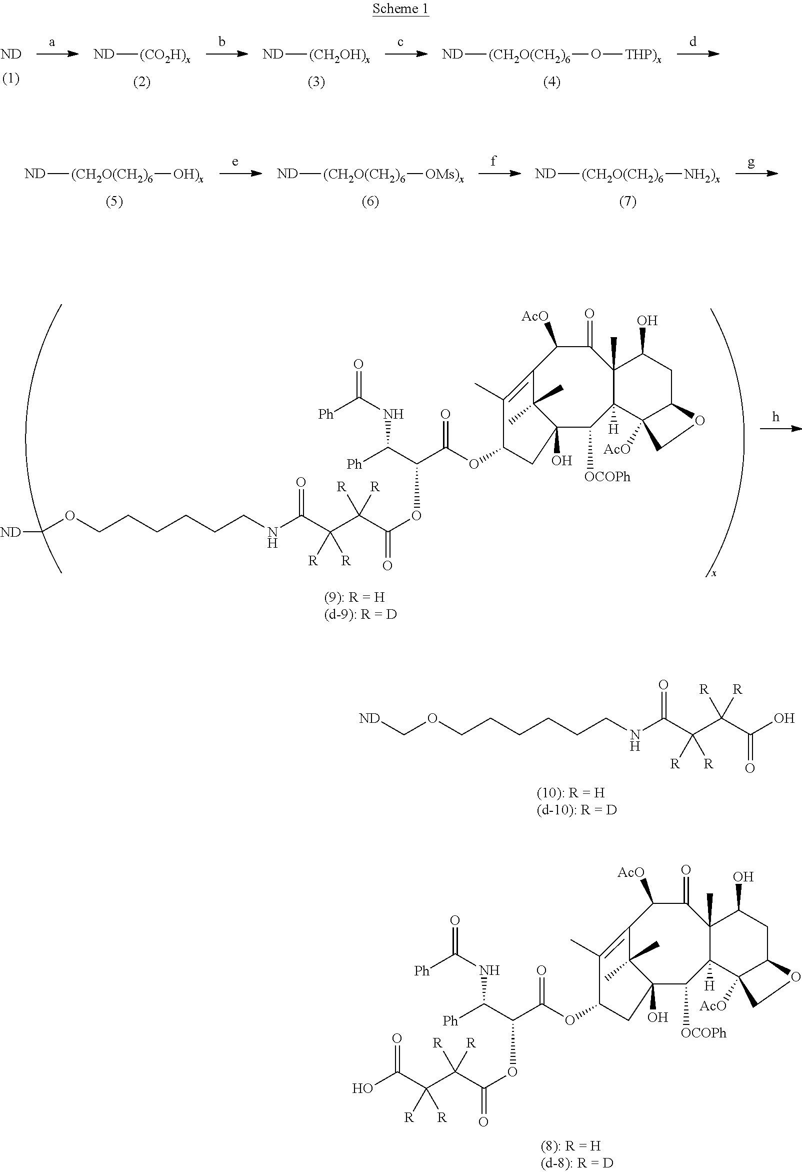

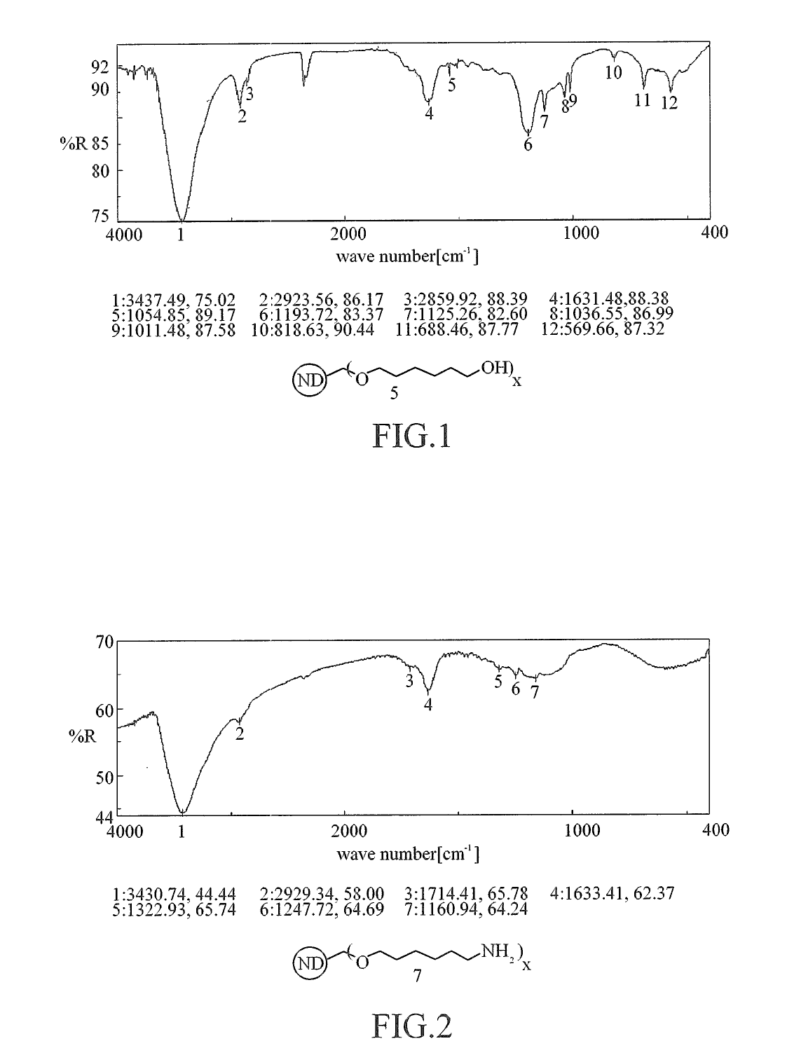

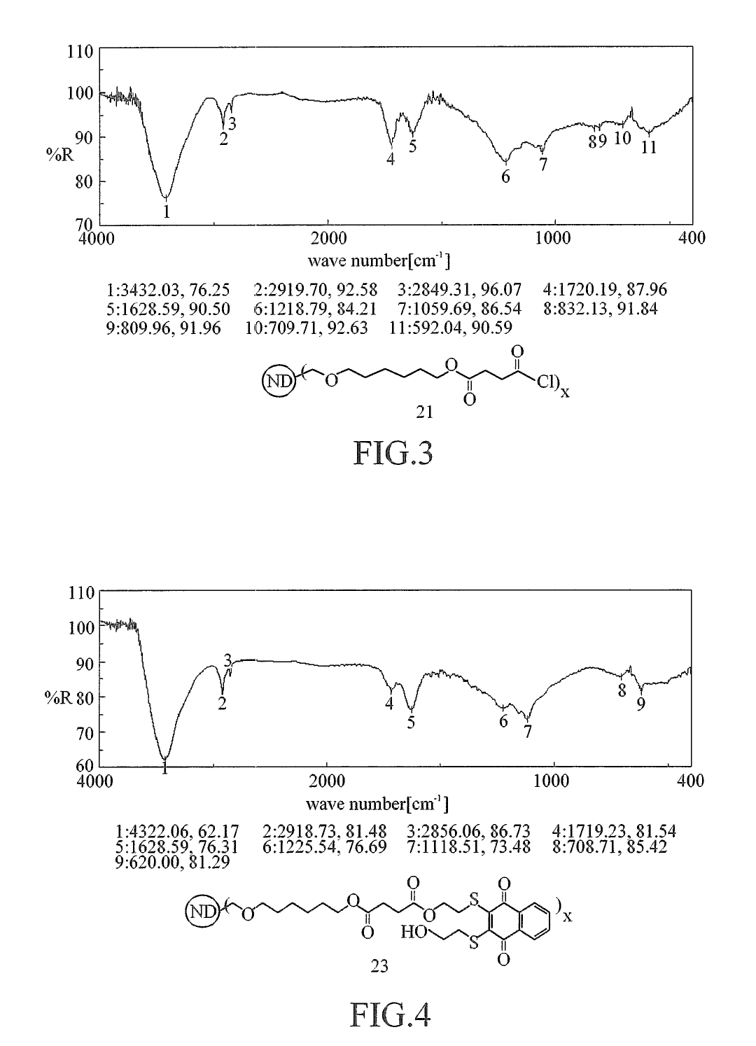

FTIR spectra of (5) and (7) in scheme 1 are shown in FIG. 1 and FIG. 2, respectively.

### Embodiment 2: Binding Iressa (ZD1839) to Nanodiamond with Linkers

Scheme 2 shows the synthesis of Iressa bound to nanodiamond. A reaction of Iressa (11) and succinic anhydride is performed to form a derivative (12) of Iressa; and a reaction of (12) and nanodiamond having linkers is performed in a solution of 2-ethoxy-1-ethoxycarbonyl-1,2-dihydroquinoline to bind derivative (12) of Iressa to nanodiamond with linkers, so as to obtain (13).

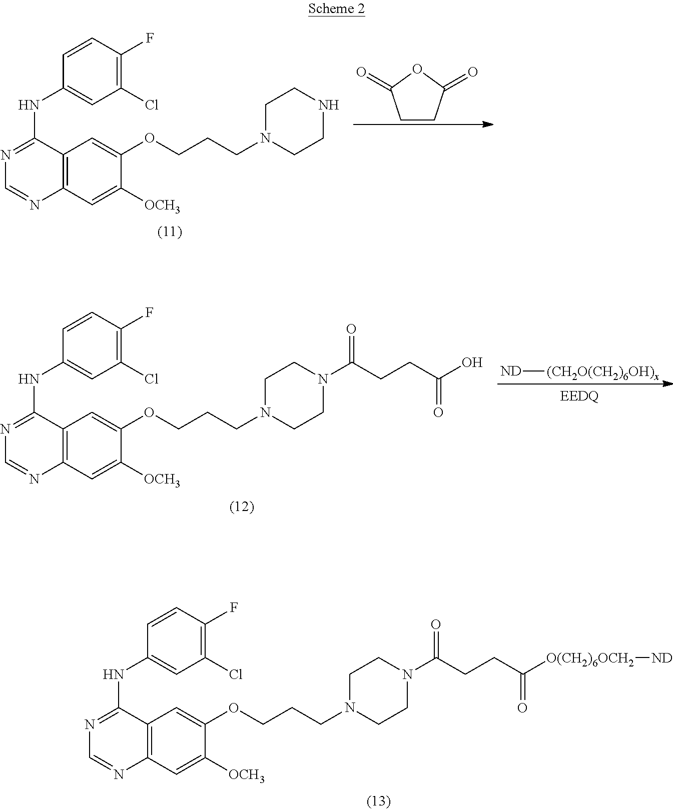

### Embodiment 3: Binding Sutent (Sunitinib) to Nanodiamond with Linkers

Scheme 3 shows the synthesis of Sutent bound to nanodiamond. A reaction of a compound (14) and a compound (15) is performed to form a compound (16), and the compound (16) is bound to a compound (17) to form a compound (18). Then, a reaction of the compound (18) and succinic anhydride is performed to form a derivate of Sutent, which is further bound to (5) to form nanodiamond bound with Sutent derivate (19).

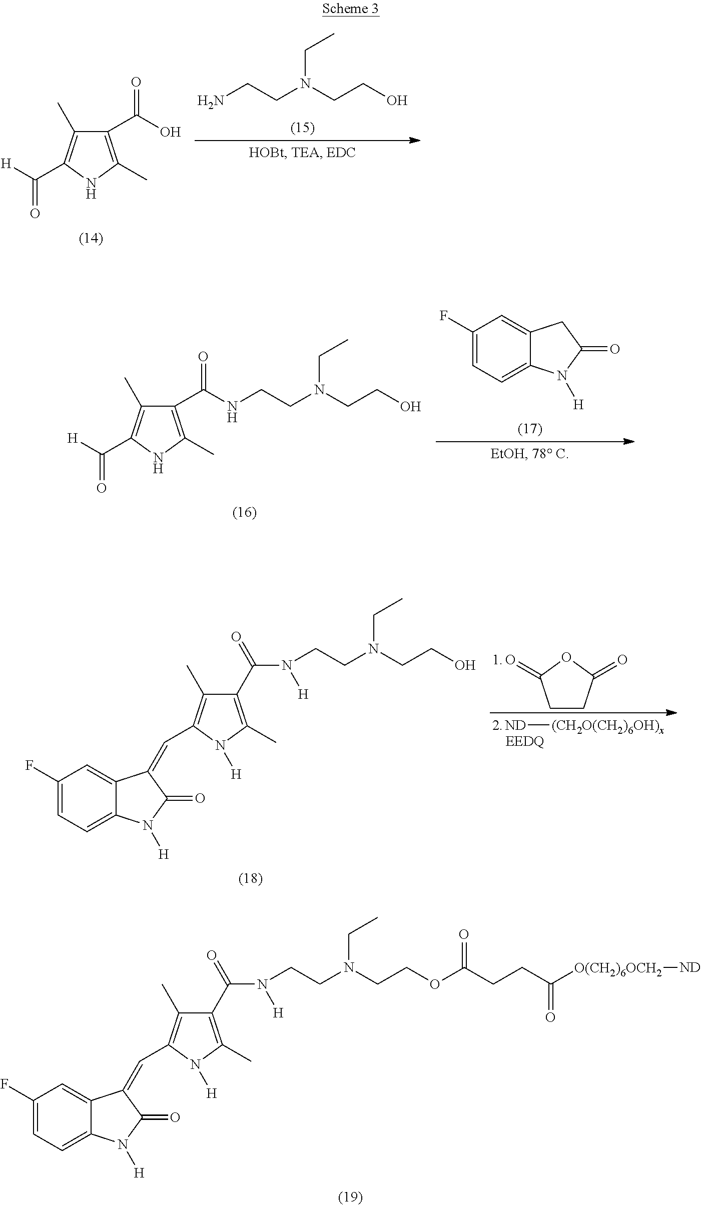

### Embodiment 4: Bonding a Vitamin K3 Derivate to Nanodiamond with Linkers

Scheme 4 shows the synthesis of a vitamin K3 derivate bound to nanodiamond. A reaction of (5) and succinic anhydride is performed to form a derivate (20) of nanodiamond, and (20) is treated with SOCl₂ to form (21), and then a reaction of (21) and a vitamin K3 derivate (22) is performed to form nanodiamond bound with the vitamin K3 derivate (23).

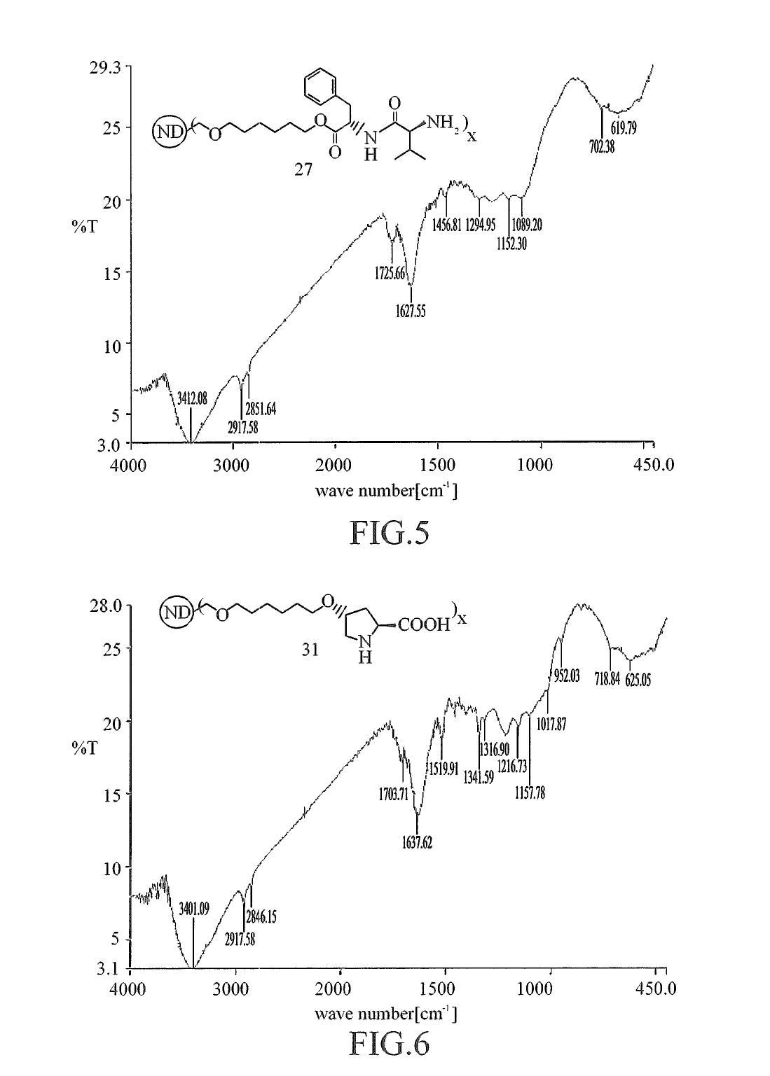

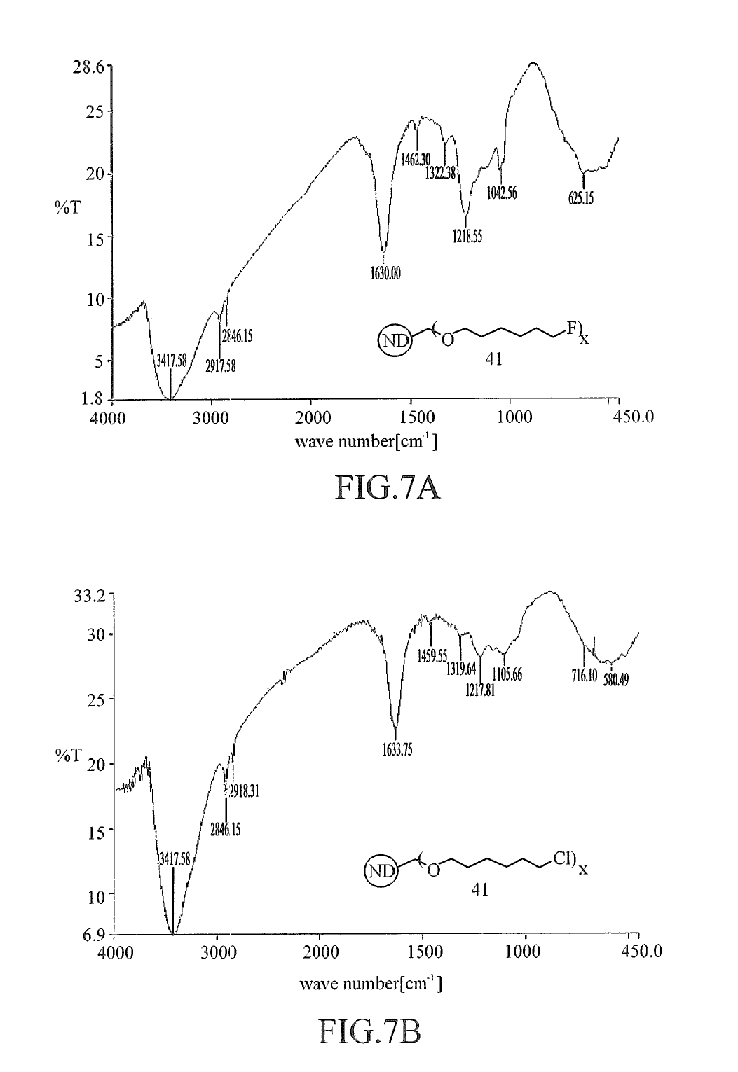

FTIR spectra of (21) and (23) in scheme 4 are shown in FIG. 3 and FIG. 4, respectively.

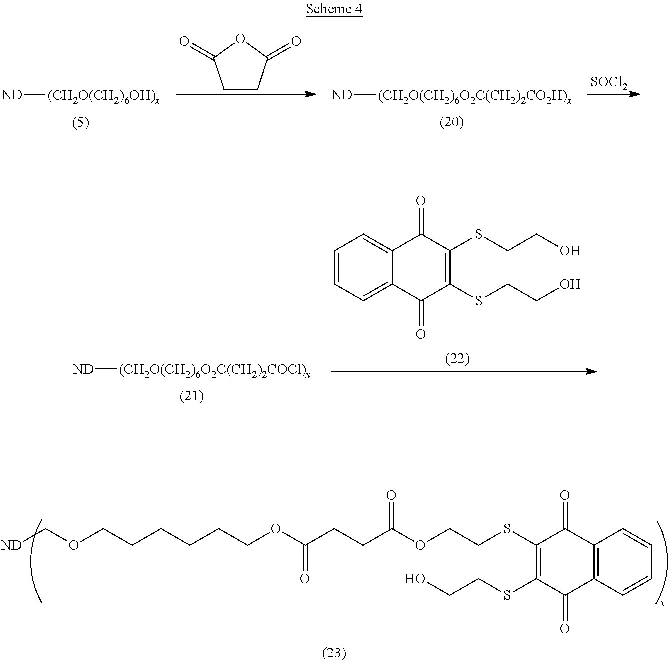

### Embodiment 5: Binding Peptides to Nanodiamond with Linkers

Scheme 5 shows the synthesis of peptides bound to nanodiamond. A reaction of (5) and N-(9-fluorenylmethoxycarbonyl)-L-phenylalanine in a mixed solution of DCC, DMAP and CH₂Cl₂ is performed to form (24). The protection group, fmoc, on (24) is cleaved by 20% of piperidine in DMF solution to form (25). Then, a reaction of (25) and N-(9-fluorenylmethoxycarbonyl)-L-valine in a mixed solution of DCC, DMAP and CH₂Cl₂ is performed to form (26). The protection group, fmoc, on (26) is cleaved by 20% of piperidine in DMF solution to form (27). (27) is treated with 1M NaOH to form dipeptide (28).

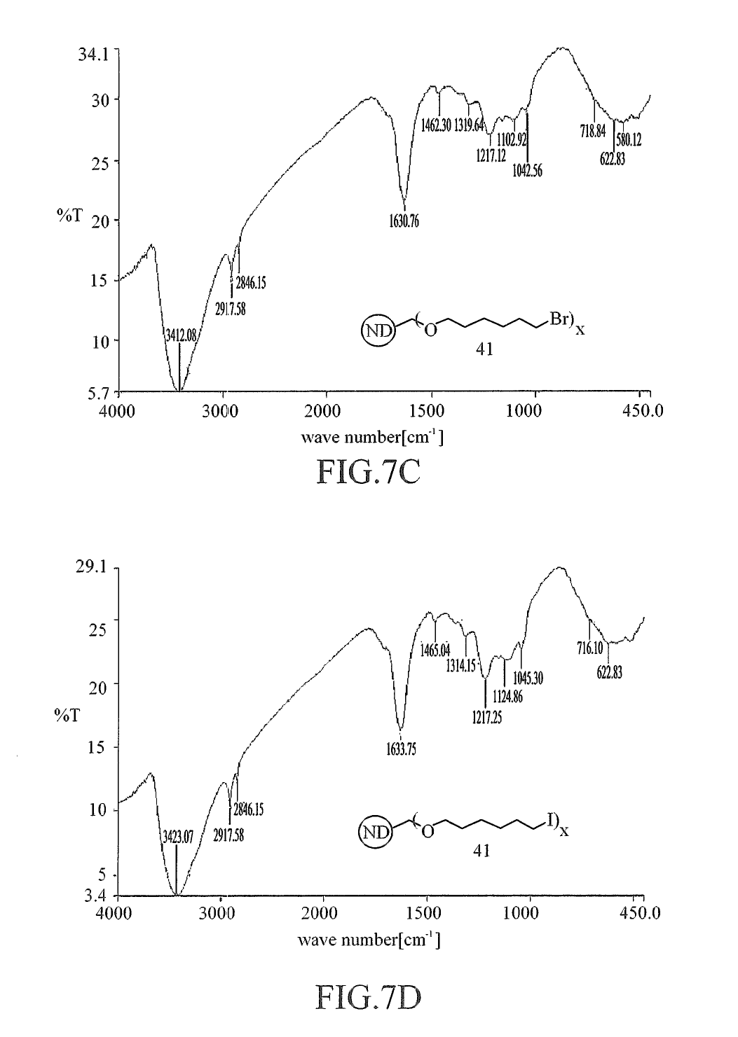

FTIR spectrum of (27) in scheme 5 is shown in FIG. 5.

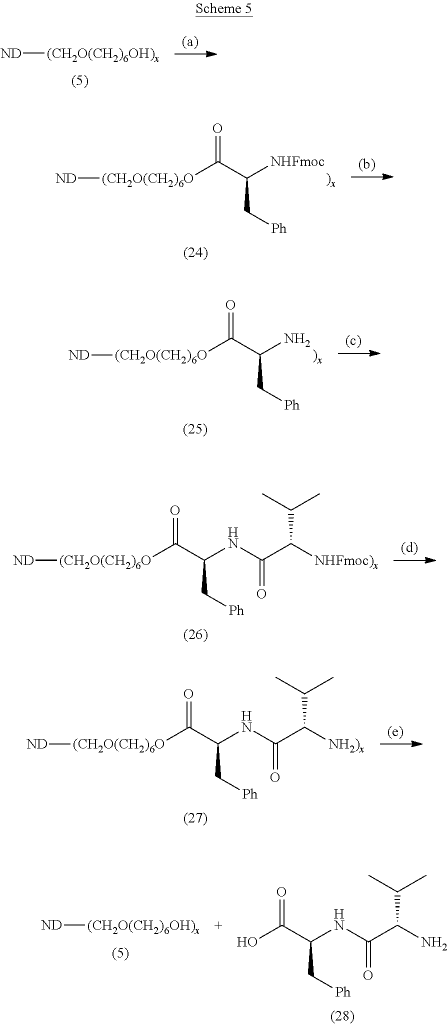

### Embodiment 6: Application of Nanodiamond Having Linkers in Asymmetry Reaction

Scheme 6 shows an application of nanodiamond having linkers in asymmetric reaction. A reaction of (5) and hydroxyproline (29) is performed to form (30), and then the protection group, Boc, of (30) is cleaved by a treatment with trifluoroacetic acid, so as to form (31), which has optical activity to be used in subsequent asymmetric reaction.

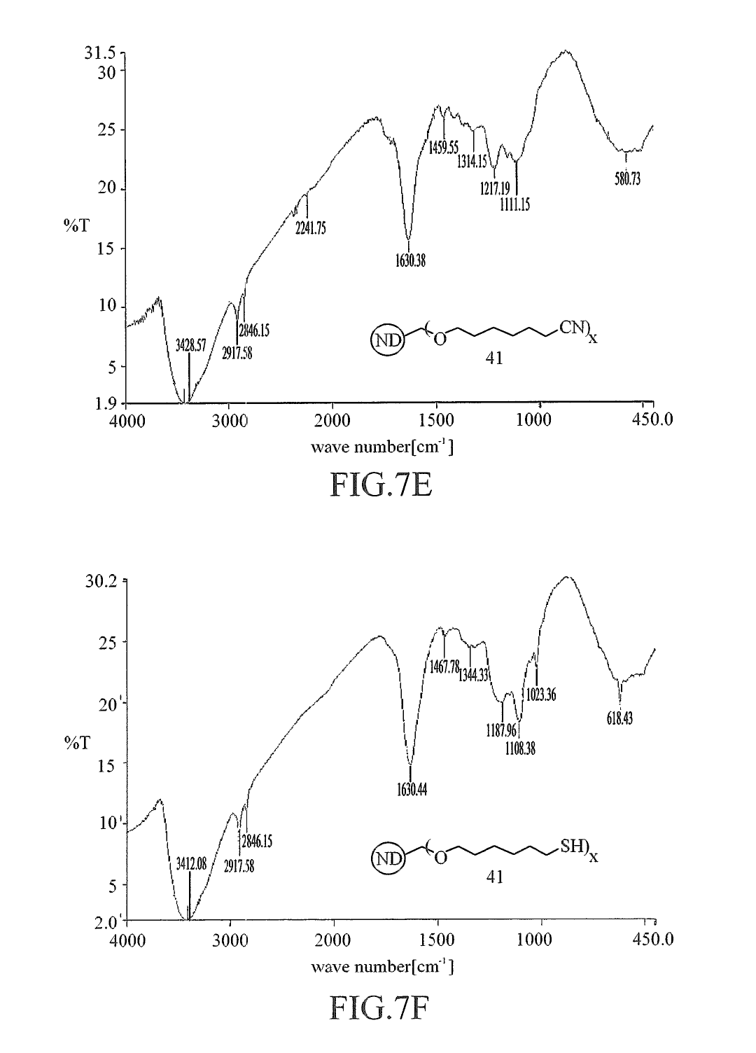

FTIR spectrum of (31) in scheme 6 is shown in FIG. 6.

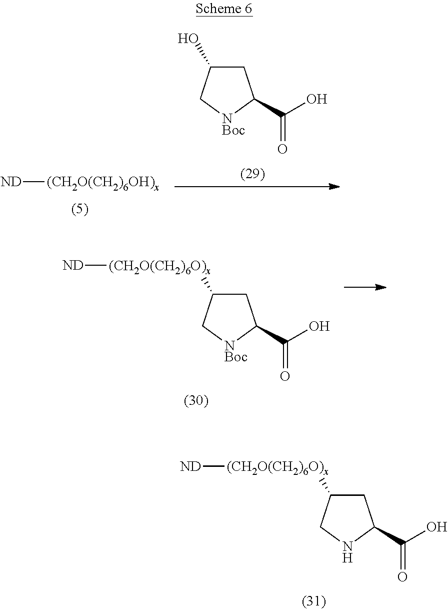

### Embodiment 7: Binding DNA to Nanodiamond with Linkers

Scheme 7 shows the synthesis of DNA bound to nanodiamond. (33) is treated with NaOH to form (33), and then (33) is treated with SOCl₂ to form (34). A reaction of (34) and DNA is performed to form nanodiamond bound with DNA (35).

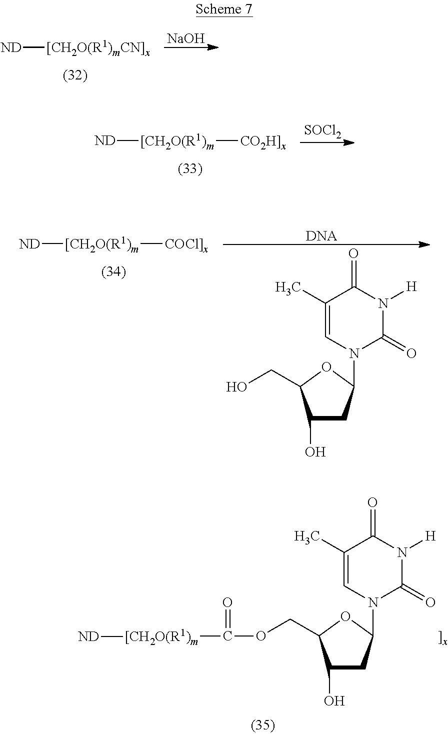

### Embodiment 8: Binding Protein to Nanodiamond with Linkers

Scheme 8 shows the synthesis of protein bound to nanodiamond. 1. Binding C-terminal of protein to nanodiamond with linkers: a reaction of (7) and protein in EDC solution is performed, so as to form (36). 2. Binding N-terminal of protein to nanodiamond with linkers: a reaction of (33) and protein in EDC solution is performed, so as to form (37).

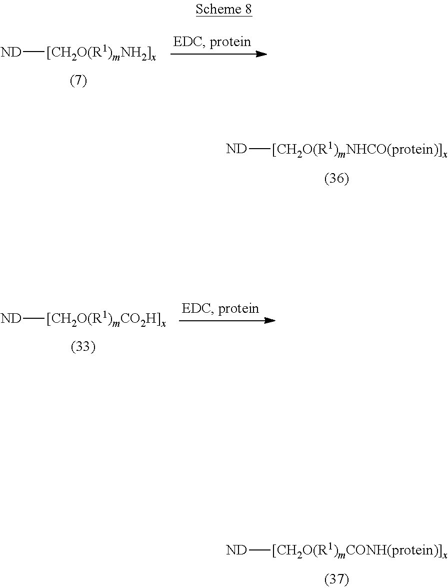

### Embodiment 9: Binding Other Synthetic Derivates to Nanodiamond with Linkers

Scheme 9 shows the synthesis of other synthetic compounds bound to nanodiamond. Nanodiamond bound with chemical derivates (R¹ to R¹²) can be bound to other drugs or biological molecules.

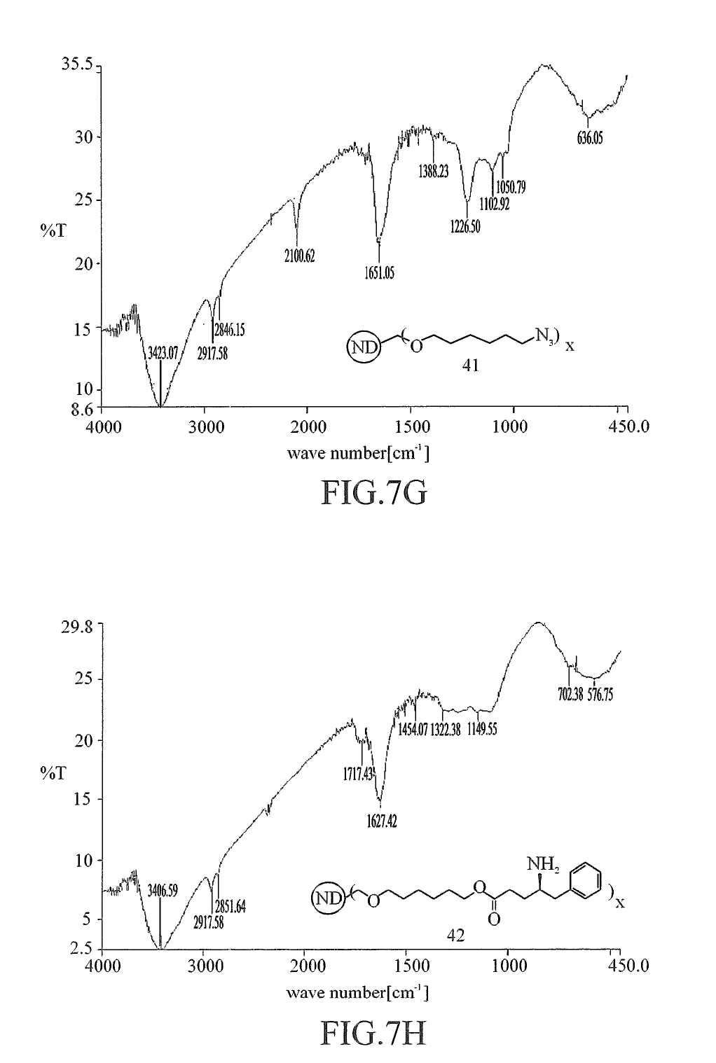

FTIR spectra of (41) and (42) in scheme 9 are shown in FIGS. 7A-7H, respectively.

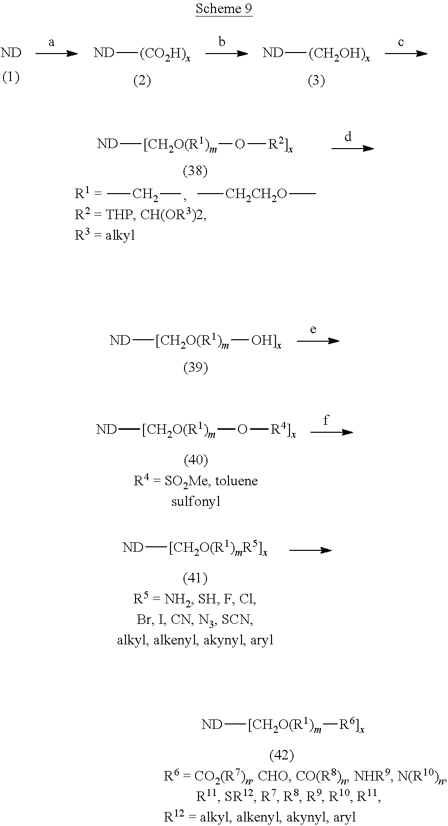

### Embodiment 10: Characterization of the Carrier Including Taxol

The bonding of the carrier including taxol obtained in embodiment 1 was determined by FTIR. The morphology and size of nanodiamond bound to taxol were also analyzed by AFM and TEM.

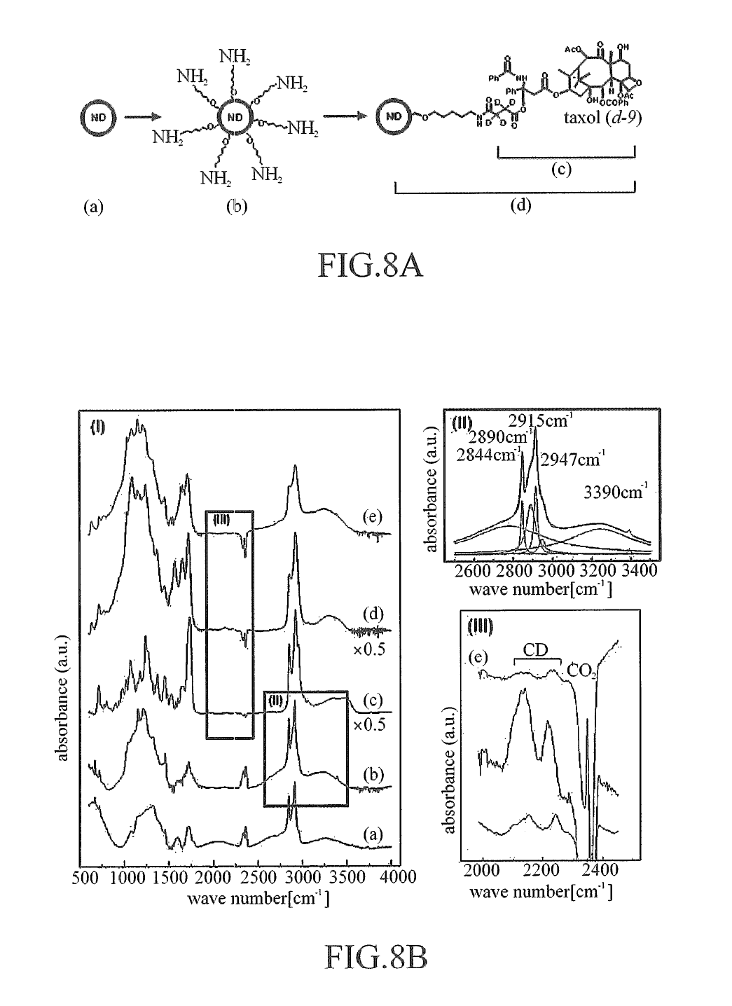

Referring to scheme 1, ND samples in each step was determined to be functionalized by using FTIR (direct absorption method), and the extension and removal of functional groups of intermediates in subsequent steps were observed. The absorbance for SO₂ is 1203 cm⁻¹ and 1315 cm⁻¹, the absorbance for NH₂ is 3390 cm⁻¹ (as shown in (b) and (II) of FIG. 8B), and the absorbance for CONH is 1700 cm⁻¹. In order to further prove that taxol is bound to the surface of nanodiamond, deuterated taxol-2′-succinic acid ester (d-8) was synthesized, and bound to the surface of nanodiamond (d-9). As shown in (b), (c), (d) and (III) in FIG. 8B, d-9 has condensed wave band at about 2131 cm⁻¹ and 2219 cm⁻¹. The ester portion of taxol is cleaved from the surface of nanodiamond by saponification of d-9, so as to leave d-10 (referring to (e) and (III) in FIG. 8B).

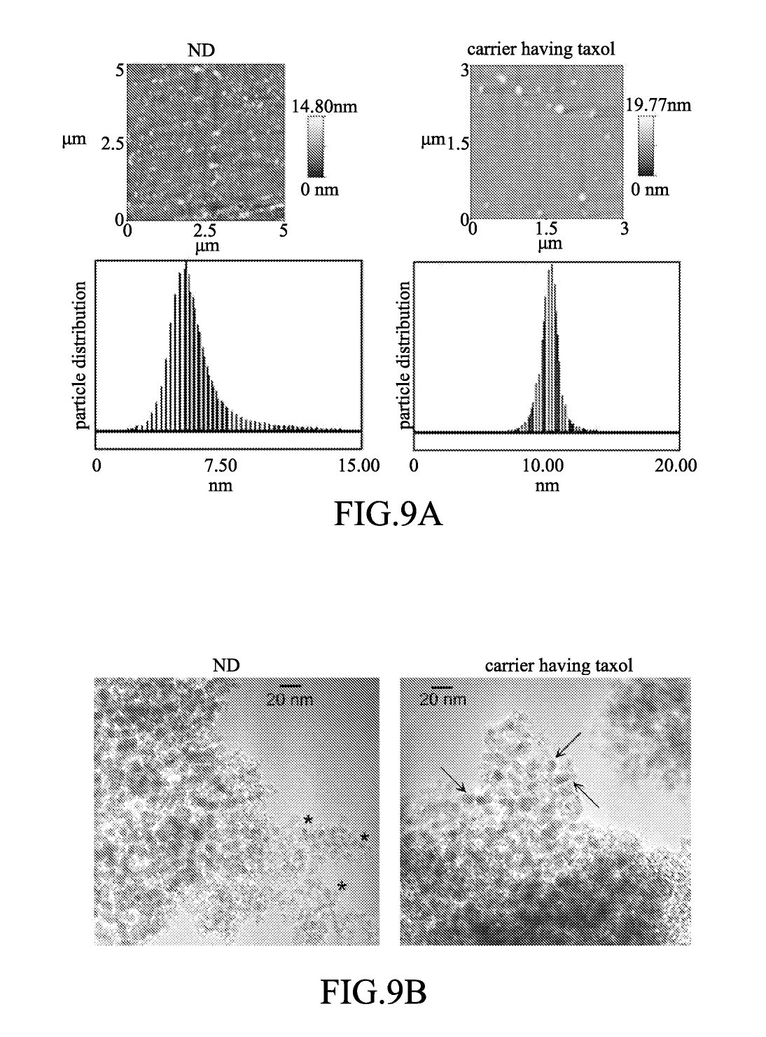

As shown in FIG. 9, granularity and surface morphology of original nanodiamond and the carrier (9) having taxol were observed by AFM and TEM. The AFM image shows the granularity of the original nanodiamond is about 5 nm (as shown in left part of FIG. 9A). The granularity of the carrier having taxol was increased to about 10 nm (as shown in right part of FIG. 9A). The TEM images of ND and the carrier having taxol are respectively shown in left part (indicated by stars) and right part (indicated by arrows) of FIG. 9B. In comparison with the original nanodiamond, the granularity of the carrier having taxol was significantly increased (referring to FIG. 9B).

### Embodiment 11: Stabilization of Microtubules and Inhibition of Mitosis by the Carrier Having Taxol

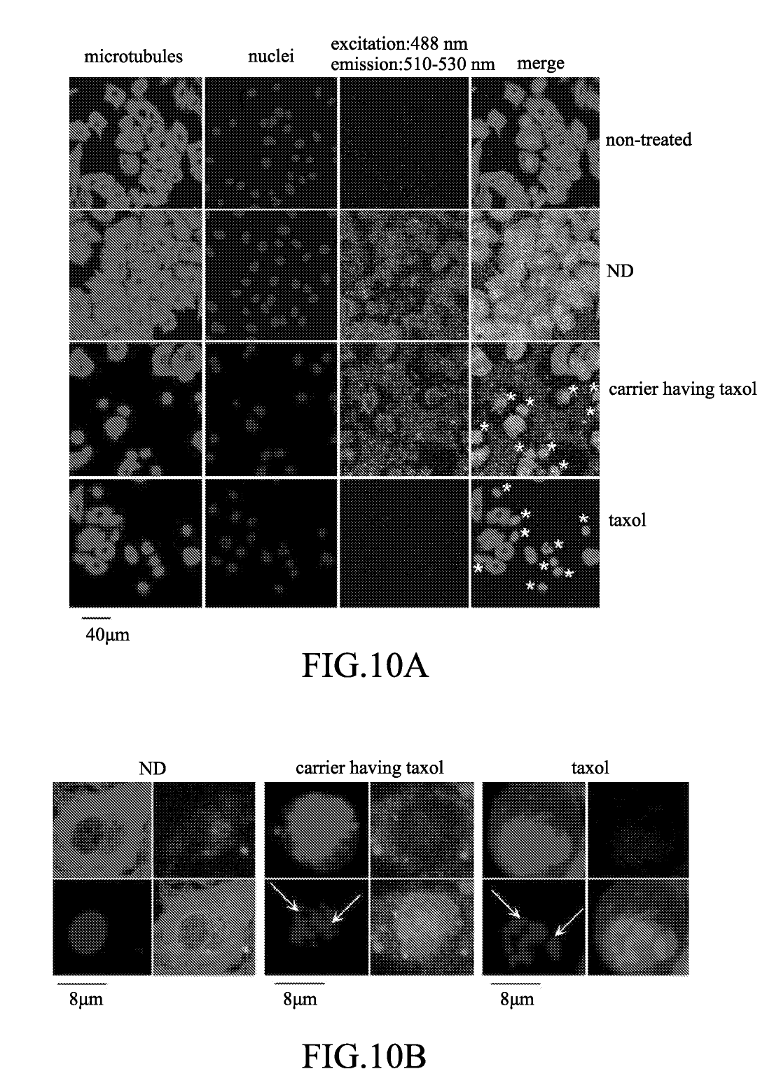

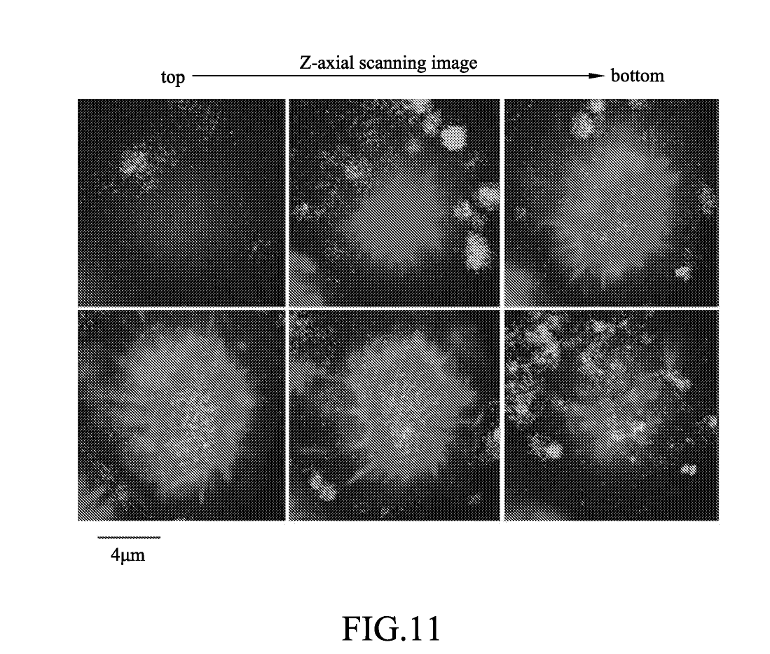

Taxol is capable of stabilizing microtubules and inducing abnormal microtubular bundles to inhibit mitosis. In order to test whether the carrier having taxol influences microtubules, A549 cells (ATCC No. CCL-185) were treated with the carrier having taxol (100 μg/mL for 24 hours), and then cytoskeleton and nuclei staining was performed on the A549 cells. Results are shown in FIG. 10. The ND particles had green fluorescence while excited at wavelength 488 nm, and the fluorescence was determined at 510-530 nm. The microtubules of A549 cells had red fluorescence (Cy3). Also, the nuclei had blue fluorescence upon Hoechst 33258 staining. The cells were treated with the carrier having taxol (100 μg/mL for 24 hours) or taxol (50 nM for 24 hours), significantly enhancing mitosis of the cells (indicated by stars in FIG. 10A. The carrier having taxol or taxol induced polymerization of microtubules, inhibited formation of spindle, and inhibited separation of chromosomes (indicated by arrows in FIG. 10B). As indicated by arrows in FIG. 10B, upon being treated with the carrier having taxol or taxol, the chromosomes of the cells were disturbed. In contrast, ND (100 μg/mL for 24 hours) alone failed to induce changes of microtubules (referring to FIG. 10A and FIG. 10B). Taxol without being bound to ND inhibited microtubules in A549 cells, but had no green fluorescence (referring to FIG. 10A and FIG. 10B). In addition, the capability for the carrier having taxol to be taken into cells was determined by Z-axial image detection using laser scanning confocal microscope, cross-section images of the A549 cells treated with the carrier having taxol (100 μg/mL for 48 hours) were captured by confocal microscope, and Z-axial cross-section image from bottom to top showed that the carriers having taxol were positioned within cells (referring to FIG. 11). The color, yellow, indicated that the carriers having taxol were positioned on microtubules.

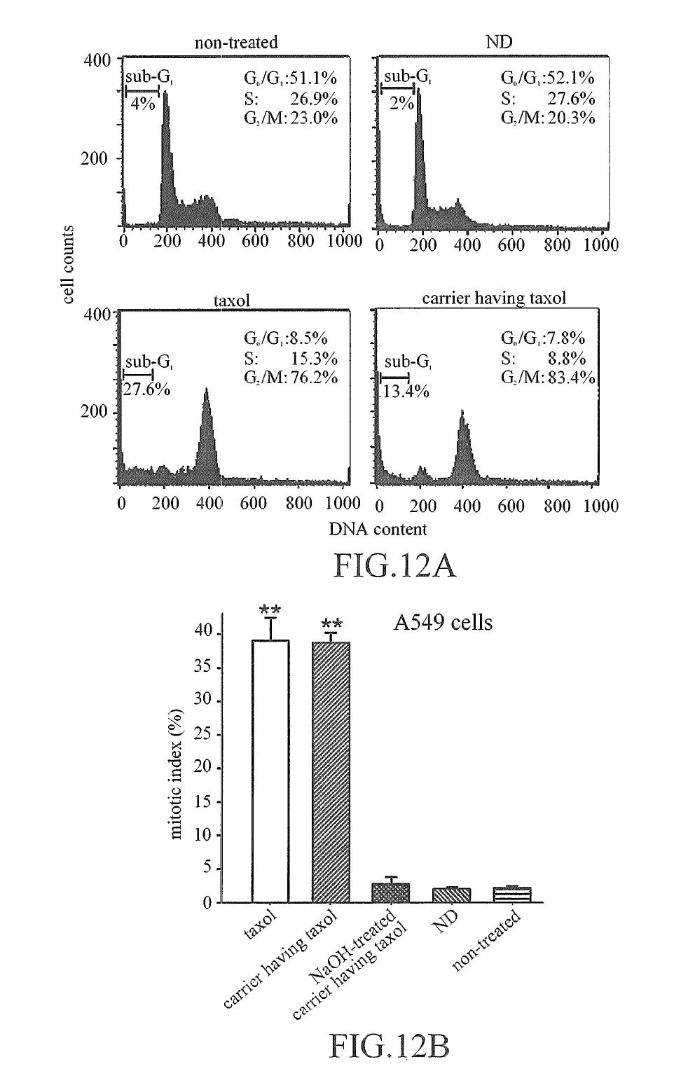

Further, the A549 cells were analyzed by flow cytometer and mitotic index, and thus the influences of the carrier having taxol on cell cycles and on termination of mitosis were determined. In comparison with the non-treated samples, the carrier having taxol or unbound taxol significantly reduced G1/G0 cell population of the A549 cells, but increased G2/M cell population (p<0.01) (referring to FIG. 12A). Upon the treatment of the carrier having taxol or unbound taxol, the G2/M cell population was respectively increased to 76.2% or 83.4%. The cells were analyzed by mitotic indexes to determine G2 or M phase induced by the carrier having taxol. Upon the treatment of the carrier having taxol (100 μg/mL for 24 hours) or unbound taxol (50 nM for 24 hours), the cells undergoing mitosis were increased to about 40% based on the number of total cells; however, ND alone did not induce the termination of mitosis on the A549 cells (referring to FIG. 12B).

In order to further prove the activity of taxol bound on ND, the carrier having taxol was treated with 1M NaOH to remove the biological activity of taxol. Upon the treatment of NaOH, the carrier having taxol did not change mitotic indexes of the A549 cells (referring to FIG. 12B).

### Embodiment 12: Induction of Cancer Cell Death and Apoptosis by the Carrier Having Taxol

In addition to inhibition of microtubules, taxol induces apoptosis. Upon triple experiments, the average of sub-G1 population (apoptotic population) induced by the carrier having taxol is 13.4% based on the number of total cells (referring to FIG. 12A). However, the sub-G1 population of the non-treated and ND-alone-treated samples was about 2-4% based on the number of total cells. There was significantly statistic difference between the sample treated by the carrier having taxol and non-treated or ND-alone-treated sample (p<0.01) (referring to FIG. 12A). The above results were obtained from three individual experiments, and the average±SE was shown in bar graph. **p<0.01 indicated that there was significantly statistic difference between the sample treated by the carrier having taxol and non-treated or ND-alone-treated sample.

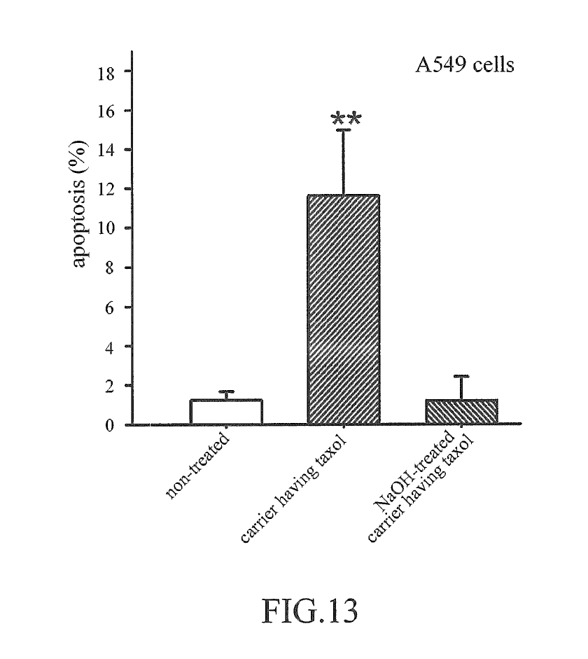

Further, by nuclei and cytoskeleton staining, the proportion of apoptotic nuclei was determined under fluorescence microscope. Similarly, the carrier having taxol significantly increased the apoptotic population (˜12%) of A549 cells (referring to FIG. 13). Upon treatment of NaOH, the carrier having taxol lost the activity to induce apoptosi of A549 cells (referring to FIG. 13). The above results were obtained from three individual experiments, and the average±SE was shown in bar graph. **p<0.01 indicated that there was significantly statistic difference between the sample treated by the carrier having taxol and non-treated or ND-alone-treated sample.

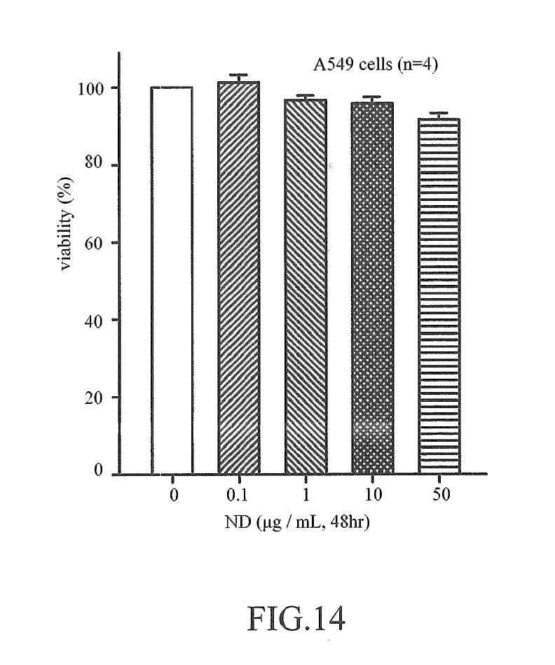

Upon the treatment with ND, the carrier having taxol, the NaOH-treated carrier having taxol, the viability of cells was analyzed by MTT. It was shown that ND particles (0.1-50 μg/mL for 48 hours) did not significantly reduce the viability of A549 cells (referring to FIG. 14). In other words, ND is a quite safe nano carbon particle. The above results were obtained from four individual experiments, and the average±SE was shown in bar graph.

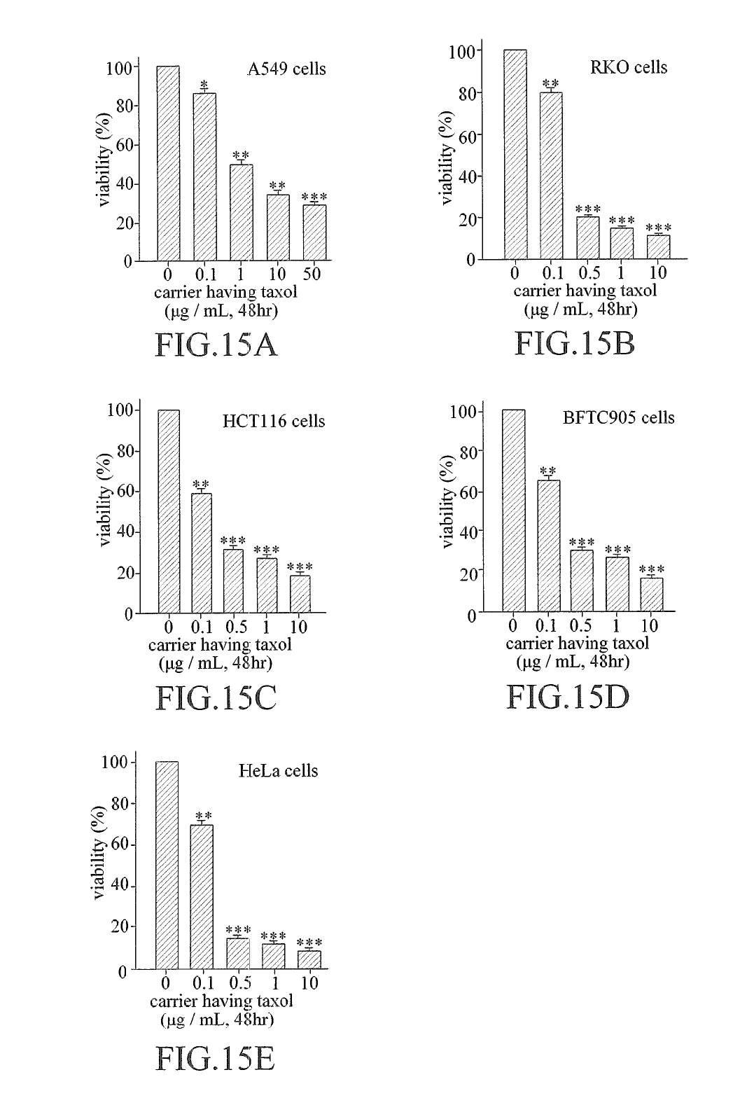

However, the carrier having taxol significantly reduces viability of various cells including A549 lung cancer cells (referring to FIG. 15A), RKO cells (colon cancer cells) (referring to FIG. 15C), BFTC905 cells (bladder cancer cells) (referring to FIG. 15D) and HeLa cells (cervical cancer cells) (referring to FIG. 15E) in a concentration-dependent manner. The above results were obtained from three to four individual experiments, and the average±SE was shown in bar graph. *p<0.05, **p<0.01 and ***p<0.001 indicated that there was significantly statistic difference between the sample treated by the carrier having taxol and non-treated sample.

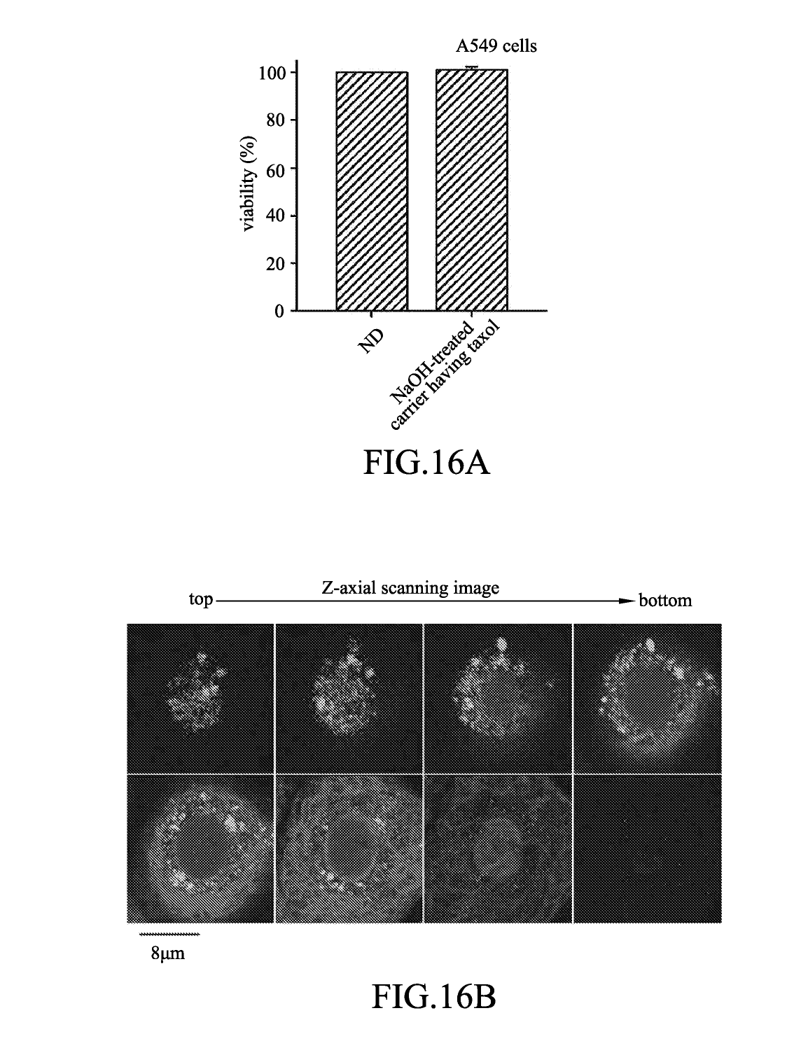

However, upon the treatment of NaOH, the carrier having taxol lost the activity to induce apoptosis of cancer cells (referring to FIG. 16A). The above results were obtained from three to eight individual experiments, and the average±SE was shown in bar graph. As shown in Z-axial confocal scanning cross-section view from bottom to top, the carrier having taxol upon treatment of NaOH was taken into cells, but did not induce damages to microtubules and nuclei (referring to FIG. 16B).

Hence, the present invention provides a carrier having taxol with anti-cancer activity for cancer therapy.

The invention has been described using exemplary preferred embodiments. However, it is to be understood that the scope of the invention is not limited to the disclosed arrangements. The scope of the claims, therefore, should be accorded the broadest interpretation, so as to encompass all such modifications and similar arrangements.

What is claimed is:

1. A carrier, comprising:
a nanodiamond particle; and
a linker covalently bound to the nanodiamond particle.

2. The carrier according to claim 1, wherein the linker is bound to a surface of the nanodiamond particle.

3. The carrier according to claim 1, wherein the linker is presented as —R¹—O(R²)ₘ-Q-,
wherein R¹ and R² are independently selected from the group consisting of a covalent bond, C₁₋₂₀alkyl, C₂₋₂₀alkenyl, C₂₋₂₀alkynyl, C₁₋₂₀alkoxy, C₁₋₂₀alkylthio and C₁₋₂₀alkylamino, in which the C₁₋₂₀alkyl, C₂₋₂₀alkenyl, C₂₋₂₀alkynyl, C₁₋₂₀alkoxy, C₁₋₂₀alkylthio and C₁₋₂₀alkylamino are optionally substituted by at least one selected from the group consisting of hydroxyl, halogen, cyano, nitro, carboxyl, C₁₋₂₀alkyl, C₂₋₂₀alkenyl, C₂₋₂₀alkynyl, C₁₋₂₀alkoxy, C₂₋₂₀alkyl ether, C₃₋₂₀alkyl ketone, C₁₋₂₀alkylthio, amino, mono-(C₁-C₂₀alkyl)amino, di-(C₁-C₂₀alkyl)amino, haloC₁₋₂₀alkyl, haloC₁₋₂₀alkoxy, C₁₋₂₀alkanoyl, C₂₋₂₀alkanoyloxy, C₁₋₂₀alkoxycarbonyl, amido (—CONH₂), mono-(C₁₋₂₀alkyl)aminocarbony, di-(C₁₋₂₀alkyl)aminocarbonyl, sulfonylamino (—SO₂NH₂), mono-(C₁₋₂₀alkyl)sulfonylamino and di-(C₁₋₂₀alkyl)sulfonylamino;
Q is hydroxyl, amino, carbonyl, acyl, keto, carboxyl, halogen, cayno, thio, C₁₋₂₀alkyl, C₁₋₂₀alkoxy, C₂₋₂₀alkenyl, C₂₋₂₀alkynyl, C₆₋₁₆aryl, azide, aldehyde, thiocyano, CO₂(R³)ₙ, CO(R⁴)ₙ, NHR⁵, N(R⁶)ₙ, SR⁷ or O(R⁸)ₙ, in which R³, R⁴, R⁵, R⁶, R⁷ and R⁸ are independently selected at each occurrence from the group consisting of halogen, amino, carbonyl, acyl, keto, carboxyl, phenylsulfonyl, sulfonyl, C₁₋₂₀alkyl, C₂₋₂₀alkenyl, C₂₋₂₀alkynyl, C₁₋₂₀alkoxy and C₆₋₁₆aryl, and n at each occurrence is an integer from 1 to 20; and
m at each occurrence is an integer from 1 to 20.

4. The carrier according to claim 3, wherein R¹ and R² are optionally substituted methyl.

5. The carrier according to claim 1, further comprising an active unit covalently bound to the linker.

7. The carrier according to claim 6, wherein the active unit is a drug, a vitamin or a biological molecule.

8. The carrier according to claim 7, wherein the drug is an anti-cancer drug.

9. The carrier according to claim 8, wherein the anti-cancer drug includes an anti-microtubule agent.

10. A carrier, comprising:
a nanodiamond particle;
at least one linker covalently bound to a surface of the nanodiamond particle; and
an active unit covalently bound to the linker.

11. The carrier according to claim 10, wherein the linker is presented as —R¹—O(R²)ₘ-Q-,
wherein R¹ and R² are independently selected from the group consisting of a covalent bond, C₁₋₂₀alkyl, C₂₋₂₀alkenyl, C₂₋₂₀alkynyl, C₁₋₂₀alkoxy, C₁₋₂₀alkylthio and C₁₋₂₀alkylamino, in which the C₁₋₂₀alkyl, C₂₋₂₀alkenyl, C₂₋₂₀alkynyl, C₁₋₂₀alkoxy, C₁₋₂₀alkylthio and C₁₋₂₀alkylamino are optionally substituted by at least one selected from the group consisting of hydroxyl, halogen, cyano, nitro, carboxyl, C₁₋₂₀alkyl, C₂₋₂₀alkenyl, C₂₋₂₀alkynyl, C₁₋₂₀alkoxy, C₂₋₂₀alkyl ether, C₃₋₂₀alkyl ketone, C₁₋₂₀alkylthio, amino, mono-(C₁-C₂₀alkyl)amino, di-(C₁-C₂₀alkyl)amino, haloC₁₋₂₀alkyl, haloC₁₋₂₀alkoxy, C₁₋₂₀alkanoyl, C₂₋₂₀alkanoyloxy, C₁₋₂₀alkoxycarbonyl, amido (—CONH₂), mono-(C₁₋₂₀alkyl)aminocarbony, di-(C₁₋₂₀alkyl)aminocarbonyl, sulfonylamino (—SO₂NH₂), mono-(C₁₋₂₀alkyl)sulfonylamino and di-(C₁₋₂₀alkyl)sulfonylamino;
Q is hydroxyl, amino, carbonyl, acyl, keto, carboxyl, halogen, cayno, thio, C₁₋₂₀alkyl, C₁₋₂₀alkoxy, C₂₋₂₀alkenyl, C₂₋₂₀alkynyl, C₆₋₁₆aryl, azide, aldehyde, thiocyano, CO₂(R³)ₙ, CO(R⁴)ₙ, NHR⁵, N(R⁶)ₙ, SR⁷ or O(R⁸)ₙ, in which R³, R⁴, R⁵, R⁶, R⁷ and R⁸ are independently selected at each occurrence from the group consisting of halogen, amino, carbonyl, acyl, keto, arboxyl, phenylsulfonyl, sulfonyl, C₁₋₂₀alkyl, C₂₋₂₀alkenyl, C₂₋₂₀alkynyl, C₁₋₂₀alkoxy and C₆₋₁₆aryl, and n at each occurrence is an integer from 1 to 20; and m at each occurrence is an integer from 1 to 20.

12. The carrier according to claim 11, wherein R¹ and R² are optionally substituted methyl.

13. The carrier according to claim 10, wherein the active unit is a drug, a vitamin or a biological molecule.

14. The carrier according to claim 13, wherein the drug is an anti-cancer drug.

15. The carrier according to claim 14, wherein the anti-cancer drug includes an anti-microtubule agent.

16. The carrier according to claim 13, wherein the vitamin is selected from the group consisting of vitamin K3, vitamin C, vitamin D, vitamin E, vitamin H and vitamin B7.

17. The carrier according to claim 13, wherein the biological molecule includes a nucleic acid, peptide, protein or derivatives thereof.

18. A use of the carrier of claim 10 for preparing a drug for treating a cancer.

19. The use according to claim 18, wherein the cancer is lung cancer, breast cancer, colorectal cancer, cervical cancer or bladder cancer.
